SEQUENCE LISTING (18 sequences)

SEQ ID NO: 1 - DNA, 17 bp, Artificial Sequence
  Primer for genotyping by PCR
tacattttgatggaggg

SEQ ID NO: 2 - DNA, 17 bp, Artificial Sequence
  Primer for genotyping by PCR
tacattttgatggaggt

SEQ ID NO: 3 - DNA, 17 bp, Artificial Sequence
  Primer for genotyping by PCR
ggaatctaatatggaag

SEQ ID NO: 4 - DNA, 17 bp, Artificial Sequence
  Primer for genotyping by PCR
tgacctggagaatctcc

SEQ ID NO: 5 - DNA, 17 bp, Artificial Sequence
  Primer for genotyping by PCR
tgacctggagaatctct

SEQ ID NO: 6 - DNA, 17 bp, Artificial Sequence
  Primer for genotyping by PCR
catccaggctctctggc

SEQ ID NO: 7 - DNA, 24 bp, Artificial Sequence
  Primer for genotyping by PCR
tgtgggctctaaagatggaactcc

SEQ ID NO: 8 - DNA, 29 bp, Artificial Sequence
  Primer for genotyping by PCR
gtgcgaggccagaggccacttgtgtagcg

SEQ ID NO: 9 - DNA, 24 bp, Artificial Sequence
  Primer for genotyping by PCR
tgtgggctctaaagatggaactcc

SEQ ID NO: 10 - DNA, 20 bp, Artificial Sequence
  Primers for real-time RT-PCR
tgtgtaaggctgcacgagtc

SEQ ID NO: 11 - DNA, 20 bp, Artificial Sequence
  Primers for real-time RT-PCR
ggcagtagcaaaaggcattg

SEQ ID NO: 12 - DNA, 20 bp, Artificial Sequence
  Primers for real-time RT-PCR
aggcttgaagacccttccat

SEQ ID NO: 13 - DNA, 20 bp, Artificial Sequence
  Primers for real-time RT-PCR
acaggcagactggtttcagg

SEQ ID NO: 14 - DNA, 14 bp, Artificial Sequence
  Primers for real-time RT-PCR
cgcccgtgtttcca

SEQ ID NO: 15 - DNA, 19 bp, Artificial Sequence
  Primers for real-time RT-PCR
tgacccatgacgtacttcc

SEQ ID NO: 16 - DNA, 17 bp, Artificial Sequence
  Primers for real-time RT-PCR
aacgacccttcattgac

SEQ ID NO: 17 - DNA, 18 bp, Artificial Sequence
  Primers for real-time RT-PCR
ccacgacatactcagcac

SEQ ID NO: 18 - protein, 82 aa, Unknown
  amino acid sequence of mouse nesfatin-1
VPIDVDKTKVHNTEPVENARIEPPDTGLYYDEYLKQVIEVLETDPHFREKLQKADIEEIRSGRLSQELDLVSHKVRTRLDEL

(19) United States
(12) Patent Application Publication
(10) Pub. No.: US 2010/0234274 A1
(43) Pub. Date: Sep. 16, 2010

(54) Use of Nestafin-1 in the Treatment for Diabetes

(76) Inventor: Jian-ning Liu, Brighton, MA (US)

Correspondence Address: LAW OFFICES OF ALBERT WAI-KIT CHAN, PLLC, 141-07 20TH AVENUE, WORLD PLAZA, SUITE 604, WHITESTONE, NY, 11357, US

(21) Appl. No.: 12/402,848

(22) Filed: Mar. 12, 2009

(51) Int. Cl. A61K 38/28; A61K 38/16; A01K 67/027; G01N 33/68

(52) U.S. Cl. 514/4; 514/12; 800/14; 800/18; 436/86

(57) ABSTRACT

The present invention provides a method of treating diabetes by increasing peripheral nesfatin-1. Peripheral nesfatin-1 can be increased by administration of nesfatin-1, conjugated nesfatin-1 that would not penetrate the blood-brain barrier, or plasmin inhibitors. The present invention also provides a method of screening for an agent that would increase peripheral or brain nesfatin-1.

### FIELD OF THE INVENTION

This invention relates generally to the study of diabetes. In one embodiment, the present invention provides a method of using nestafin-1 to treat diabetes.

### BACKGROUND OF THE INVENTION

Although the plasminogen system is primarily responsible for fibrin degradation, its roles in brain and neurological function have been implicated. Plasminogen and its activators (TPA and uPA) are expressed in developing/adult brains, including hippocampal large pyramidal neurons and dendrites. Plasmin was reported to be involved in the process of hormones derived from the POMC precursor in the intermediate pituitary.

Brain hypothalamus expressed several secreted molecules that function in regulating feeding behavior. NUCB2/nucleobindin 2 (also called NEFA for DNA binding/EF-hand/acidic protein) is a hypothalamus-secreted protein containing 396 amino acids that is highly conserved in human, mice and rat. Polypeptide encoded by the NEFA gene has a calcium-binding domain (EF domain) and a DNA-binding domain. NEFA has a high homology with nucleobindin and is considered to be a member of the DNA-binding factor called the EF-hand superfamily having reactivity with calcium.

NUCB2 when injected directly into the brain of rats promotes anorexia and decreases body weight. NUCB2 is cleaved posttranslationally by pro-hormone convertases into an N-terminus-fragment Nesfatin-1 (NEFA/nucleobindin2-encoded satiety- and fat-influencing protein) and two C-terminal peptides, Nesfatin-2 and Nesfatin-3. Nesfatin-1 possesses all of the anorexigenic property of NUCB2. Intracerebroventricular (i.c.v.) or i.p. injection of nesfatin-1 inhibits food intake and thereby reduces body weight. The conversion of NUCB2 into Nesfatin-1 is indispensable for its activity in vivo. Nesfatin-1 is found in discrete nuclei of the hypothalamus where it probably activates a leptin-independent melanocortin pathway. Nesfatin-1 crosses the Blood Brain Barrier (BBB) in both the blood-to-brain and brain-to-blood directions by a nonsaturable system.

NUCB2 is also expressed in the adipocyte cell line 3T3L1 suggesting other functions of Nesfatin-1 outside brain or peripheral source of Nesfatin-1 affecting brain function. Nesfatin-1 in rat stimulates calcium influx and interacts with a G protein-coupled receptor still to be characterized.

Carefully examining the amino acid sequence of nesfatin-1, it is highly conserved from mouse to human and has several putative cleavage sites by plasmin. Therefore, it is of interest to determine whether nesfatin-1 could mediate the effect of plasminogen in obese and diabetic animals.

### SUMMARY OF THE INVENTION

Nesfatin-1 was previously reported as a satiety molecule to suppress food intake via the melanocortin signaling in hypothalamus. Here it is reported that nesfatin-1 improved diabetic symptoms peripherally in db/db mice in addition to its central inhibition of appetite. Based on a postulation that nesfatin-1 was the putative substrate of plasmin, plasminogen and leptin receptor or leptin gene double deficient mice were generated to investigate the effect of elevated nesfatin-1 in obese and diabetic animals. The double knockouts had significantly higher hypothalamic nesfatin-1, less food intakes and lighter body weights than their counterparties, db/db and ob/ob. The high blood glucose and insulin in db/db were normalized by plasminogen deficiency. Nesfatin-1 was found surprisingly abundant in serum, >2500-fold more than hypothalamus, and always more with freely feeding than fasting. Interestingly, the cerebral TPA was also found lower with freely feeding than fasting, related to the proteolytic inactivation of nesfatin-1. Peripheral nesfatin-1 was also believed to be degraded by plasmin at least in-part, evidenced by the following two findings: one was that intravenous administration of AMCA and aprotinin had similar effects to plasminogen knockout in db/db, and another was that i.v. nesfatin-1 was cleared much slower in plg^{-/-} than plg^{+/+} mice. Peripheral injection of nesfatin-1 significantly reduced blood glucose in db/db. Since the effect of nesfatin-1 was insulin-dependent, it is promising to be developed into a novel therapeutics for type-II diabetes.

### BRIEF DESCRIPTION OF THE FIGURES

Data were presented as means ±SEM as indicated in the figure legends. All data were representative of at least three different experiments. Comparisons between individual data points were made using a two-tailed student's t-test. Differences were considered statistically significant when p was less than 0.05.

FIG. 1 shows (a) genotyping of the littermates from crossbreeding of plg^{+/-} and lepr^{+/-} mice (left), and from plg^{+/-} and lep^{+/-} mice (right). The 268 bp and 190 bp-bands corresponded to the plasminogen wild-type and targeted alleles, respectively. Both wild-type and mutant alleles of leptin receptor or leptin were 400 base pairs. (b) Body weight of plg^{+/+} lepr^{-/-} and db/db mice over 24 weeks on chow diet. Number of mice used showed in parentheses.

FIG. 2 shows body weight of mice over 24 weeks on chow diet and their daily food intake at 5, 10, 15 and 20 weeks of age, (FIG. 2a, c) plg/lepr and (FIG. 2b, d) plg/lep. Error bars indicated SEM. *, p<0.05 compared with obese mice.

FIG. 3a shows fasting blood glucose of mice over ages. FIG. 3b shows serum insulin of mice aged 12 weeks. FIG. 3c shows Blood glucose during IGTT of mice aged 16-18 weeks. Data represented the mean ±SEM. *, p<0.05, **, p<0.01 compared with obese mice. Number of mice used showed in parentheses.

FIG. 4 shows (a) the amino acid sequence of mouse nesfatin-1. Arrows indicated the putative cleavage sites by plasmin. (b) Nesfatin-1 was completely digested by plasmin within 2 hours.

FIG. 5 shows the hypothalamic nesfatin-1 measured by HPLC in (a) each genotype; (b) with fasting and freely feeding. Data represented the mean ±SEM for samples in quadruplicate (three mouse hypothalami per sample).

FIG. 6 shows hypothalamic mRNA encoding neuropeptides in plg^{-/-}lepr^{-/-} mice versus littermates including plg^{+/+}lepr^{-/-}. The mRNA Expression was normalized to gadph. (a) agrp, (b) npy and (c) pomc, all measured by quantitative real-time PCR. Data represented the mean ±SEM for samples in triplicate.

FIG. 7 shows (a) SDS-PAGE zymography of mouse hypothalamus extracts: control-TPA standard, ff-free feeding, f-fasting; (b) Serum nesfatin-1 in mice; (c) Blood glucose of mice injected with nesfatin-1/saline; (d) The dose-dependent and (e) time-dependent effect of i.v. nesfatin-1; (f) The IGTT in wild-type mice; (g) The effect of nesfatin-1 in the Streptozotocin-induced type-I diabetic C57BL/6J mice, 4 males/group. Data represented the mean ±SEM (*, p<0.05, **, p<0.01). Number of mice used showed in parentheses.

FIG. 8 shows (a) serum nesfatin-1 in db/db at 30 minutes after i.v. AMCA and aprotinin. Lower nesfatin-1 in “saline” than “ff db/db” (FIG. 3b) was due to blood dilution after injection. (b) Reduction in body weight after a 3-day i.v. AMCA (15 mg/day); (c) Food intake at day 3 during a 3-day i.v. AMCA (15 mg/day). Number of mice used showed in parentheses.

FIG. 9 shows immunohistochemistry of AgRP in arcuate of (a) plg^{+/+}lepr^{+/+}; (b) plg^{-/-}lepr^{+/+}; (c) plg^{-/-}lepr^{-/-}; and (d) plg^{+/+}lepr^{-/-}.

FIG. 10 shows blood nesfatin-1 was measured with HPLC (loading of 25 μl serum from free fed wild-type mouse).

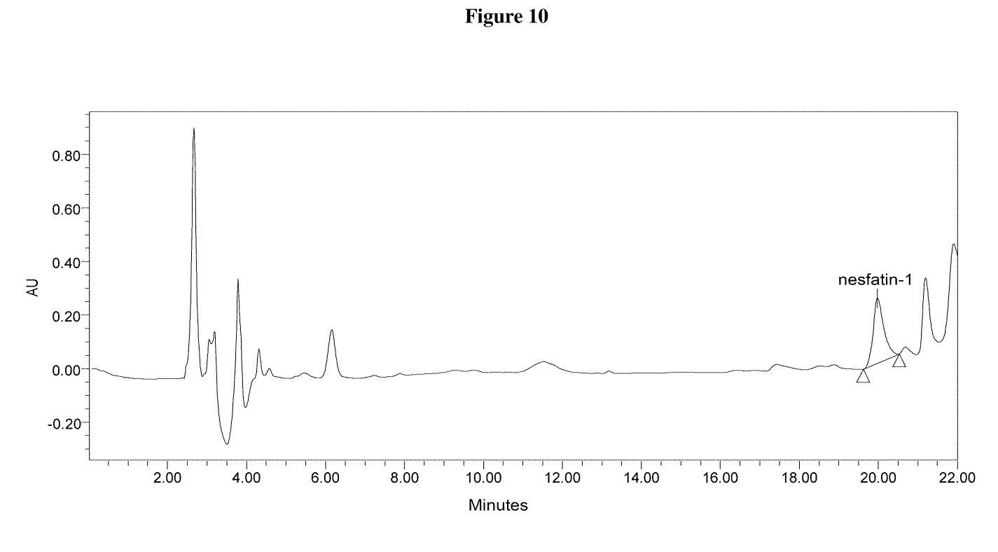

### DETAILED DESCRIPTION OF THE INVENTION

The present invention provides a method of treating a subject having diabetes, comprising the step of administering to the subject an effective amount of an agent that leads to increased peripheral nesfatin-1. In general, the agent can be administered intravenously, subcutaneously, or orally. In one embodiment, the agent is nesfatin-1 or a portion thereof. The nesfatin-1 can be human or rodent nesfatin-1. Using standard methodology in the art, one of ordinary skill in the art would readily determine a portion or domain of nesfatin-1 that manifests the activity of nesfatin-1. For example, truncated nesfatin-1 or fragments of nesfatin-1 can be generated by standard recombinant techniques and tested in the assays described herein to determine their anti-diabetic activities. Moreover, recombinant mutants of nesfatin-1 can also be tested. The present method covers the use of molecules which contain full-length nesfatin-1, a portion thereof, or a mutant nesfatin-1. In one embodiment, the molecule is a polypeptide.

In another embodiment, the agent is a conjugated nesfatin-1 having increased molecular weight. One of ordinary skill in the art would readily construct a higher molecular weight nesfatin-1 by conjugating nesfatin-1 with a number of carriers or proteins well-known in the art such as albumin, immunoglobulin, Fc, Apo-lipoprotein, etc. Such conjugated nesfatin-1 would reduce blood glucose without penetration of blood-brain barrier.

In another embodiment, the agent for the above method can be a plasmin inhibitor. Examples of plasmin inhibitor include, but are not limited to, aprotinin, AMCA (tranexamic acid), EACA (epsilon-amino-caproic acid) or their analogues.

The above method would be useful for treating a subject having type II diabetes. In another embodiment, the method would be useful for treating a subject having type I diabetes, wherein treatment for type I diabetes would further comprise the step of administering insulin to the subject. In another embodiment, the above method also results in reduced body weight or reduced food intake in the subject.

The present invention also provides uses of an agent that increases peripheral nesfatin-1 for the treatment of diabetes. Examples of agents that would increase peripheral nesfatin-1 have been discussed above. Such uses would be useful for treating type II diabetes, or treating type I diabetes together with the administration of insulin.

The present invention also provides a transgenic diabetic or obese rodent comprising homozygous plasminogen gene disruption, wherein the transgenic rodent exhibits reduced body weight or reduced blood glucose as compared to a diabetic or obese rodent not having the plasminogen gene disruption. In one embodiment, the transgenic rodent further comprises homozygous leptin gene disruption or homozygous leptin receptor gene disruption. In one embodiment, the transgenic rodent is a mouse. Such transgenic animals would be useful in a number of studies such as drug screening, clearance studies for nesfatin-1, etc.

The present invention also provides a method of reducing triglyceride, total cholesterol or LDL in blood, comprising the step of administering to a subject an effective amount of an agent that leads to increased peripheral nesfatin-1. Examples of agents that would increase peripheral nesfatin-1 have been discussed above.

The present invention also provides a method of screening for an agent that would increase peripheral or brain nesfatin-1, comprising the steps of: (i) administering a candidate agent to a subject; (ii) obtaining blood samples or brain tissue samples from the subject; and (iii) determining the amount of nesfatin-1 in the samples, wherein an increased amount of nesfatin-1 as compared to that in samples obtained from subject treated with a control substance would indicate that the candidate agent would increase peripheral or brain nesfatin-1. In one embodiment, the subject in the screening method is the transgenic rodent described above. In one embodiment, the amount of nesfatin-1 can be determined by a HPLC assay as described herein. In another embodiment, the amount of nesfatin-1 can be determined by a number of assays that utilize anti-nesfatin-1 antibodies (e.g. ELISA assay).

The invention being generally described, will be more readily understood by reference to the following example which are included merely for purpose of illustration of certain aspects and embodiments of the present inventions, and are not intended to limit the invention.

Throughout this application, various references or publications are cited. Disclosures of these references or publications in their entireties are hereby incorporated by reference into this application in order to more fully describe the state of the art to which this invention pertains.

### EXAMPLE 1

### Generation of Plasminogen Deficient Obese or Diabetic Mice

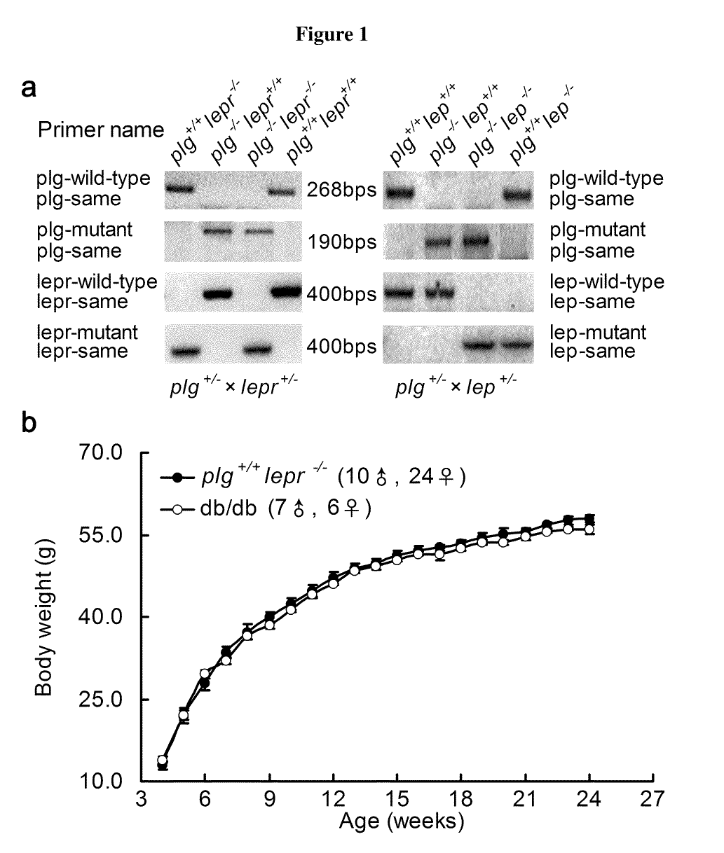

Lepr^{+/-} mice in C57BLKS/J, plasminogen heterozygous (plg^{+/-}) and lep^{+/-} in C57B1/6J were purchased from Jackson Laboratory (Bar Harbor, Me.) and raised in our laboratory. All animals were kept under specific pathogen-free conditions with lab chow available adlibitum in a 12-hour light/dark cycle. Plg^{+/-} mice were crossed with lepr^{+/-} and lep^{+/-} to generate plg^{+/-}lepr^{+/-} and plg^{+/-}lep^{+/-} mice. These mice were then used to produce plasminogen deficient in db/db and ob/ob mice, plg^{-/-}lepr^{-/-} and plg^{-/-}lep^{-/-}. Mice (lepr wild-type, lepr mutant, lep wild-type, lep-mutant, plasminogen wild-type, plasminogen mutant) were genotyped by PCR using genomic DNA isolated from tail tips (FIG. 1a). The primer sequences were used as following, lepr-wild-type-F: 5′-TAC ATT TTG ATG GAG GG-3′ (SEQ ID NO:1); lepr-mutant-F: 5′-TAC ATT TTG ATG GAG GT-3′ (SEQ ID NO:2); lepr-same-R: 5′- GGA ATC TAA TAT GGA AG -3′ (SEQ ID NO:3); lep-wild-type-F: 5′-TGA CCT GGA GAA TCT CC-3′ (SEQ ID NO:4); lep-mutant-F: 5′-TGA CCT GGA GAA TCT CT-3′ (SEQ ID NO:5); lep-same-R: 5′-CAT CCA GGC TCT CTG GC-3′ (SEQ ID NO:6); plg-wild-type-F: 5′-TGT GGG CTC TAA AGA TGG AAC TCC-3′ (SEQ ID NO:7); plg-mutant-F: 5′-GTG CGA GGC CAG AGG CCA CTT GTG TAG CG-3′ (SEQ ID NO:8); plg-same-R: 5′-TGT GGG CTC TAA AGA TGG AAC TCC-3′ (SEQ ID NO:9).

The plg^{+/+}lepr^{-/-} and db/db weighed same over the age (FIG. 1b) and this indicated that the cross did not impact the body weight of db/db mice.

### EXAMPLE 2

### Characteristics of Plasminogen Deficient Obese or Diabetic Mice

Mice without leptin or leptin receptor are obese, diabetic, infertile, hyperphagic and hypoactive. As reported herein, plasminogen deficiency not only significantly reduced obesity in both mice, but also dramatically improved diabetic symptoms in db/db.

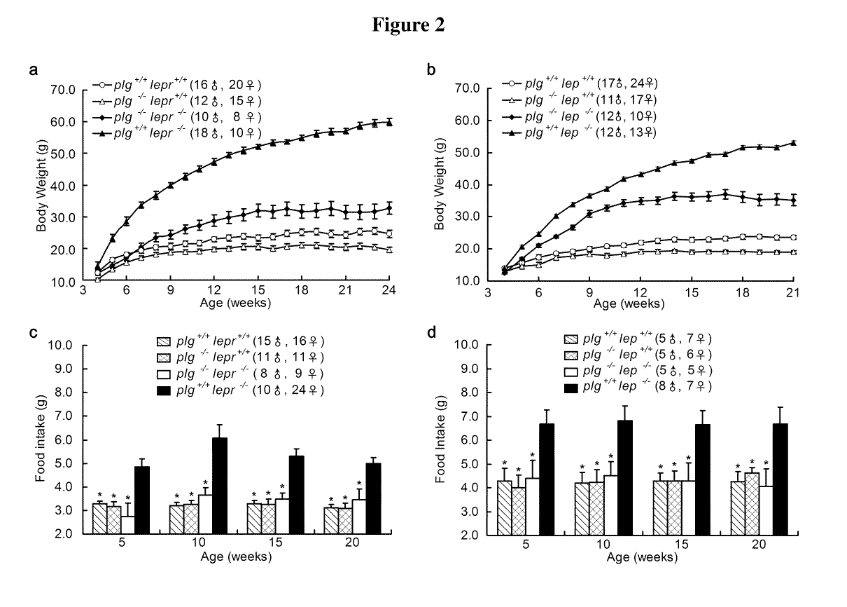

Mice were placed on standard lab chow and followed for >24 weeks. As body weight was measured weekly, the chow consumed was recorded daily and averaged over the week. As shown in FIG. 2, the body weights and food intakes of plg^{-/-}lepr^{-/-} and plg^{-/-}lep^{-/-} were markedly reduced on chow diet, compared to their obese littermates, while plg^{+/+}lepr^{-/-} and db/db weighed same over the age.

### Assay for Blood Glucose/Insulin and the Intraperitoneal Glucose Tolerance Test (IGTT)

Mice of the investigated genotype (plg^{+/+}lepr^{-/-}, plg^{-/-}lepr^{-/-}, plg^{+/+}lepr^{+/+}, plg^{-/-}lepr^{+/+}) were fasted for 18 hours at 8, 16 and 24 weeks old, before blood sampling from tail veins to measure fasting blood glucose using a glucose meter (Accu-Chek, Roche). Mice serum insulin was measured at the age of 12 weeks by ELISA (ALPCO, Salem, N.H.). IGTT was performed previously. As briefly, 12-week old mice were placed in clean cages without food at 4 pm on the day prior to the experiment. At 10 am the following day, the mice were injected intravenously with 1 mg glucose per gram of body weight. Blood glucose was measured immediately before and at 10, 20, 30, 60, 90, 120, and 180 minutes after the injection of glucose.

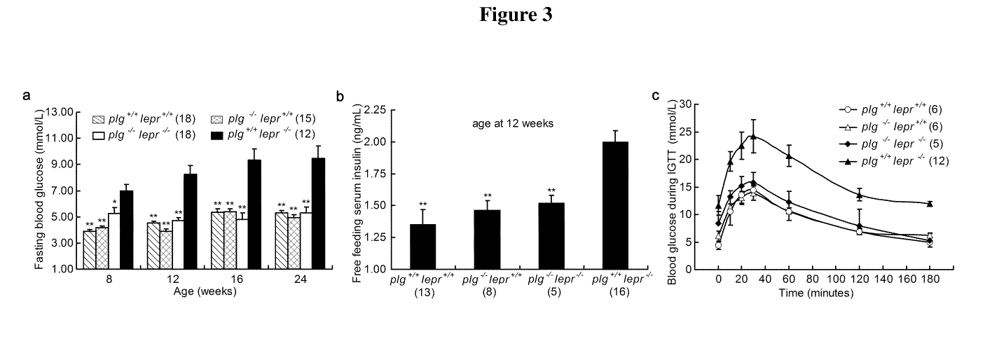

The high blood glucose (FIG. 3a) and insulin (FIG. 3b) of db/db were normalized in plg^{-/-}lepr^{-/-}. In the intraperitoneal (i.p.) glucose tolerance test (IGTT), plg^{-/-}lepr^{-/-} mice reacted normally to injected glucose contrary to diabetic plg^{+/+}lepr^{-/-} (FIG. 3c). As reported herein, plasminogen deficiency not only significantly reduced obesity in db/db and ob/ob mice, but also dramatically improved diabetic symptoms in db/db.

### EXAMPLE 3

### Nesfatin-1 Mediates The Effects of Plasminogen in Obese and Diabetic Animals

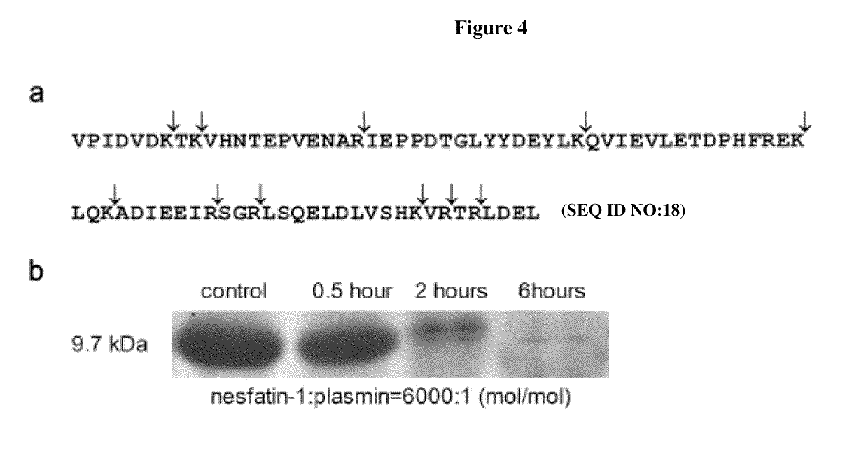

Nesfatin-1, a secreted fragment of NUCB2, has been recently identified as an anorexigenic factor associated with melanocortin signaling in hypothalamus. The intracerebroventricular (i.c.v.) or i.p. injection of nesfatin-1 inhibits food intake and thereby reduces body weight. Carefully examining the amino acid sequence of nesfatin-1, it is highly conserved from mouse to human and has several putative cleavage sites by plasmin (FIG. 4). Therefore, it is postulated that nesfatin-1 could mediate the effect of plasminogen in obese and diabetic animals.

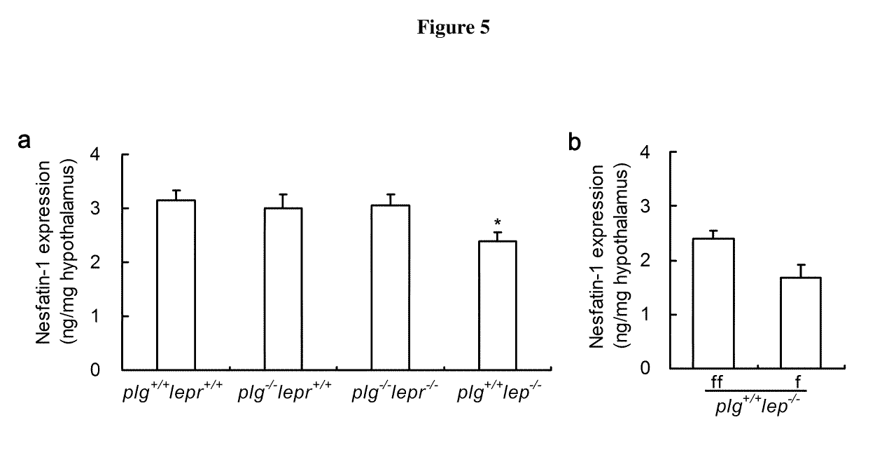

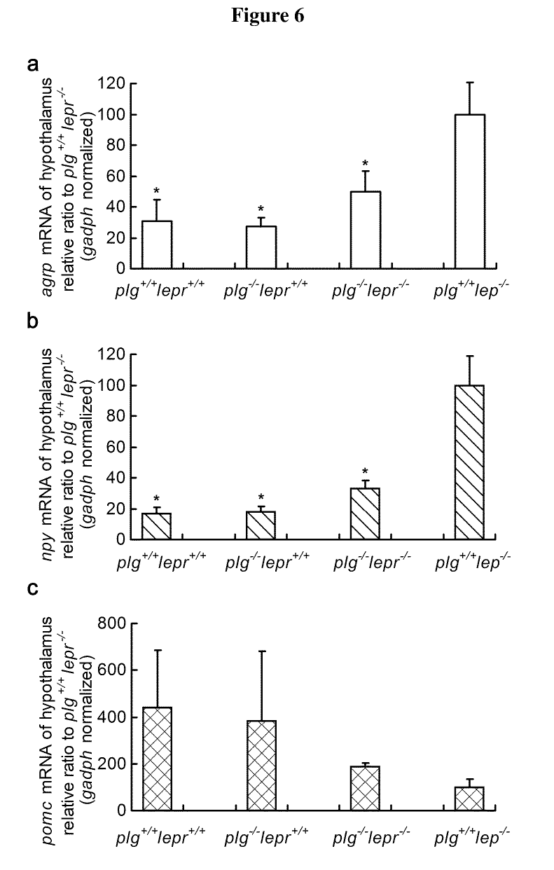

To prove this assumption, recombinant nesfatin-1 was expressed and purified from genetically engineered E. coli. It was then incubated with plasmin and rapidly degraded as expected (FIG. 4b). Intriguingly, hypothalamic nesfatin-1 was found significantly less in plg^{+/+}lepr^{-/-} than their non-obese littermates included plg^{-/-}lepr^{-/-} (FIG. 5), suggesting at least in-part plasminogen/plasmin was accounted for the decrease in nesfatin-1. The recovery of hypothalamic nesfatin-1 in plg^{-/-}lepr^{-/-} was consistent to its normalization in food intake and body weight (FIG. 2ac). Fasted mice always had less nesfatin-1 in hypothalamus than their freely feeding counterparties (FIG. 5b). Furthermore, plasminogen deficiency provided a unique approach to study the chronic effect of elevated nesfatin-1 in hyperphagic obese animals, which was impossible for the i.c.v. or i.p. administration due to the short half-time of nesfatin-1. Consequently, agrp and npy were found reduced in plg^{-/-}lepr^{-/-}, while pomc was elevated compared with plg^{+/+}lepr^{-/-} (FIG. 6), contradicting to the previous report that i.c.v. nesfatin-1 didn't alter the expression of pomc, npy and agrp. Consistent to the present finding, the same group reported the up-regulation of pomc by i.p. nesfatin-1 in NTS recently.

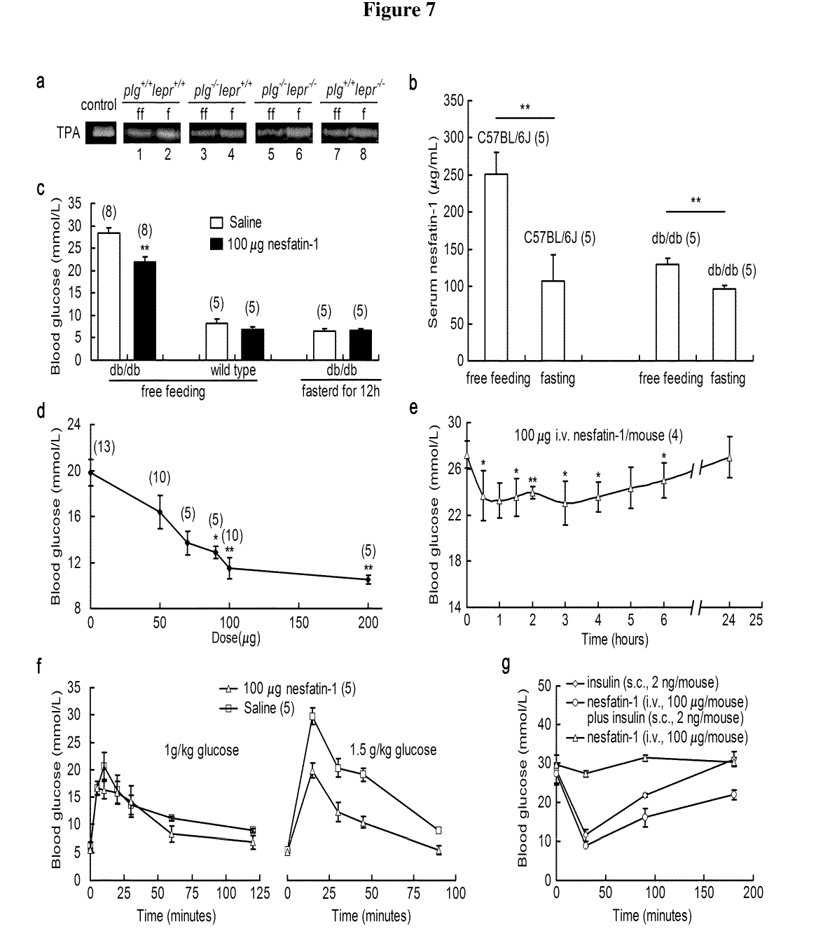

Logically, the proteolytic reduction in nesfatin-1 requires plasmin generation. Indeed, the activity of tissue plasminogen activator (TPA) was increased in hypothalamus from freely feeding to fasting (FIG. 7a), well correlated to the changes in hypothalamic nesfatin-1 (FIG. 5). For the first time, the TPA/plasminogen system was indicated to be involved in the feeding behavior, which was previously suggested to be involved in the learning process. It is well known that feeding can facilitate learning in animals. Nesfatin-1 appears to connect these two important brain functions.

Surprisingly, it was found that there were >2500-fold more nesfatin-1 in mouse serum than hypothalamus (FIG. 7b & FIG. 5), which was also reduced from freely feeding to fasting. Amazingly as described above, the diabetic symptoms of db/db mice were essentially eliminated in plg^{-/-}lepr^{-/-}, which couldn't be possibly explained by the anorexigenic effect of nesfatin-1. The db/db mice with fasting, caloric restriction or i.c.v. injection of nesfatin-1 had little diabetic improvement (data not shown), suggesting that the anti-diabetic effect by plasminogen deficiency would be peripheral rather than neurological. The i.v. administration of 100 μg nesfatin-1 significantly reduced blood glucose in freely fed db/db, but not in fasted db/db and lean wild-type mice (FIG. 7c). This anti-diabetic effect was dose- and time-dependent (FIG. 7d, e). Although the half-life of nesfatin-1 was 10 minutes in circulation, its effect lasted >6 hours, suggesting an enduring intracellular mechanism. During IGTT using wild-type mice, 100 μg nesfatin-1 significantly enhanced the uptake of blood sugar with i.v. injection of 1.5 g/kg glucose, but not 1 g/kg (FIG. 7f). Because nesfatin-1 only reduced blood glucose at the high dose inducing insulin secretion, its effect would be insulin-dependent. Indeed, in the Streptozotocin-induced type-I diabetic mice, the blood glucose decreased only when nesfatin-1 was injected with s.c. insulin (FIG. 7g).

### Methods

### Zymography Assay for the Activity of Tissue Plasminogen Activator (TPA) in Hypothalamus

Zymography was used to determine the activity of TPA in hypothalamus as described previously². Hypothalamus isolated from mouse with or without fasting was homogenized and centrifuged. Samples normalized by equal quantity of proteins were mixed with the sample buffer and loaded onto 10% SDS-polyacrylamic gel containing 3 mg/ml casein and 4.5 mg/ml plasminogen. Human TPA 0.1 ng (Genentech, San Francisco, Calif.) was used as a positive control. Following electrophoresis, the gels were soaked in a renature buffer (0.02% NaN3, 200 mM NaCl, 50 mM Tris-HCl, 2.5% Triton X-100, pH 8.3) for 30 minutes at room temperature, and then incubated in the developing buffer (0.02% NaN3, 200 mM NaCl, 50 mM Tris-HCl, pH 8.3) at 37° C. for 18 hours. To visualize the lysis band of TPA, the gels were stained with Coomassie Brilliant Blue R-250 and then destained until clear bands appeared on the blue background.

### HPLC Assay for Serum Nesfatin- 1

Hypothalamus in acetic acid supplemented with protease inhibitor cocktail tablets (Roche, Indianapolis, Ind.) was homogenized, sonicated and heated at 95° C. for 15 minutes. The samples were then centrifuged at 13,200 rpm at 4° C. for 30 minutes. The supernatants were finally collected as their protein contents were determined by the Bradford assay (Thermo-Fisher Sci. Rockford, Ill.). Mouse serum was freshly prepared by drawing blood through ophthalmectomy. About 100 mg hypothalamic total proteins or 25 μL serum were analyzed with Waters Delta 600E/2487/717 HPLC system using an analytical C18 reverse phase column (4.6×250 mm/5 μm, Hambon, Zhangjiagang, Conn.). Nesfatin-1 was eluted with a linear gradient from 20%-40% solvent B (solvent A: water with 0.1% trifluoroacetic acid, solvent B: acetonitrile with 0.1% trifluoroacetic acid) for 20 minutes at the flow rate of iml/minute. The purified nesfatin-1 was used as the standard to determine the retention time and plot the standard curve. The fraction collected at the retention time was sent for the mass spectrometry analysis.

### Streptozotocin-Induced Type-I Diabetic Mice

Male C57BL/6J mice (10 weeks) were given intraperitoneal injections of Streptozotocin (STZ) in sodium citrate (pH 4.5) on two consecutive days (100 mg/kg/day). Blood glucose was measured by tail vein sampling using the glucose oxidase enzymatic test. Diabetes was defined as a morning blood glucose reading of >16 mM after STZ. When blood glucose levels exceeded 30 mM, diabetic mice were given 16 ng of porcine insulin (Wangbang, Xuzhou, Conn.) every second day to prevent weight loss while maintaining blood glucose levels within the hyperglycemic range (16-30 mM). Nesfatin-1 (100 μg/mouse) was i.v. injected either alone or combined with s.c. insulin (2 ng/mouse) to STZ-induced type-I diabetic mice.

### EXAMPLE 4

### The Effect of Intravenous Injection of Plasmin Inhibitor on Blood Nesfatin-1

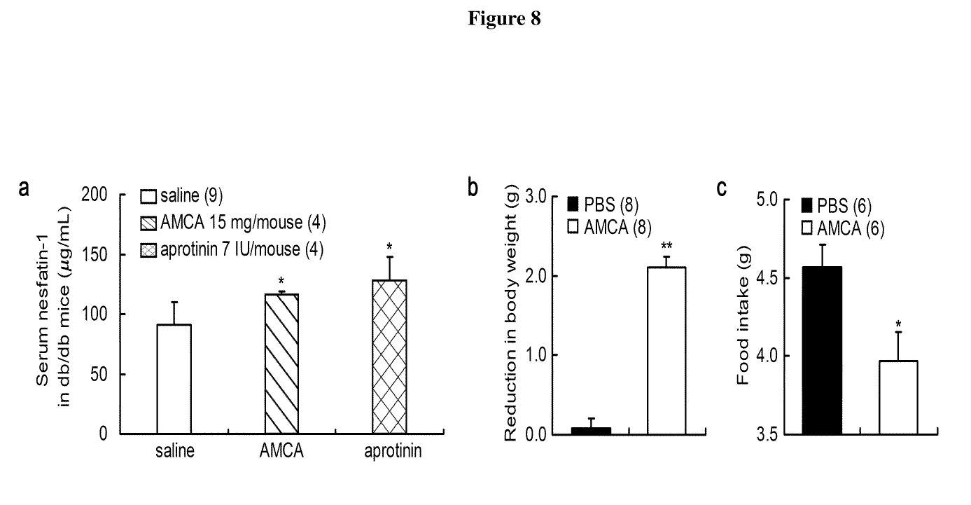

It is unknown how nesfatin-1 is cleared from circulation. The tiny amount of plasmin generation in periphery was previously reported and confirmed in our study (data not shown). AMCA and aprotinin, two inhibitors of plasmin, was i.v. injected to db/db. The reduction in food intake and body weight was seen while circulating nesfatin-1 was increased (FIG. 8). The i.v. nesfatin-1 also cleared much slower in plg^{-/-} than plg^{+/+} (data not shown). Therefore, peripheral nesfatin-1 was believed at least partially to be degraded by plasmin. Consistent to the report that nesfatin-1 penetrated BBB without saturation, the anorexigenic effect of AMCA suggested that peripheral nesfatin-1 was at least in-part of the source of cerebral nesfatin-1. Since nesfatin-1 was found to affect rats neuropsychologically as evidenced by increasing anxiety and fear-related behaviors, albumin-nesfatin-1 fusion protein were made effectively reducing blood glucose without entering the brain (data not shown).

For the first time, it was found that TPA/plasminogen directly affects the homeostasis of energy expenditure including appetite, body weight and blood sugar through its proteolytic inactivation of nesfatin-1, although it was found to affect adipocyte differentiation previously. More importantly, the data presented herein demonstrate the anti-diabetic effect of peripheral nesfatin-1, which could lead to a novel treatment for type-II diabetes.

### EXAMPLE 5

### Quantitative PCR Assay for Neuropeptides

The neuropeptide mRNA was measured using quantitative PCR (q-PCR), using CFX96TM Real-Time System (Bio-Rad, Hercules, Calif.) and the SYBR Green I detection method. Briefly, hypothalamic tissues from 24-hour fasted mice were homogenized, and total RNA was extracted using RNAiso Reagent (TaKaRa, Dalian, Conn.) and then reversed to single-strand cDNA. The relatively expression of neuropeptide mRNA was determined using the standard curves of hypothalamic cDNA, and adjusted for total RNA contents with gadph RNA by qPCR. Primers for real-time RT-PCR were used as follows: agrp forward primers: 5′-TGT GTA AGG CTG CAC GAG TC (SEQ ID NO:10); agrp reverse primers: 5′-GGC AGT AGC AAA AGG CAT TG (SEQ ID NO:11); agrp Tm: 61 ° C.; npy forward primers: 5′-AGG CTT GAA GAC CCT TCC AT (SEQ ID NO:12); npy reverse primers: 5′-ACA GGC AGA CTG GTT TCA GG (SEQ ID NO:13); npy Tm: 61° C.; pomc forward primers: 5′-CGC CCG TGT TTC CA (SEQ ID NO:14); pomc reverse primers: 5′-TGA CCC ATG ACG TAC TTC C (SEQ ID NO:15); pomc Tm: 58° C.; gadph forward primers: 5′- AAC GAC CCC TTC ATT GAC (SEQ ID NO:16); gadph reverse primers: 5′- TCC ACG ACA TAC TCA GCA C (SEQ ID NO:17); gadph Tm: 60° C. All the samples were run in triplicate, and the results were averaged.

### EXAMPLE 6

### Immunohistochemistry of AgRP on Hypothalamus

After 48-hour fasting, mouse was deeply anesthetized with sodium pentobarbital and transcardially perfused with 20 ml saline, followed by 50 ml of 4% paraformaldehyde in PBS (pH7.4). The brain was removed and post fixed overnight, then stored in PBS with 30% sucrose. To measure the immunofluorescence of AgRP, cryostat sections (20 μm thick) were post fixed with paraformaldehyde, incubated with 1% BSA in PBS for 20 minutes, and then with rabbit anti-AgRP antibody (1:4000, Phoenix Pharmaceuticals, Burlingame, Calif.) in the same solution for 1 days at 4° C. After being washed three times in PBS, the sections were incubated with Cy2-conjugated goat anti-rabbit IgG (1:250, Jackson, West Grove, Pa.) for 2 hours at room temperature, and then washed three times in PBS, mounted and cover-slipped with the buffered glycerol (pH8.5).

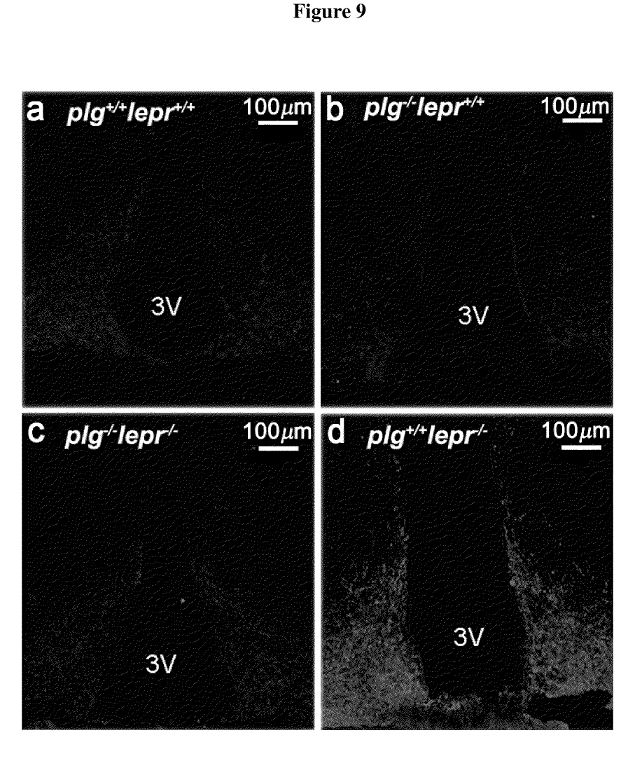

As shown in FIG. 9, The hypothalamic agrp and npy were found reduced in plg^{-/-}lepr^{-/-}, while pomc was elevated compared with plg^{+/+}lepr^{-/-}.

### EXAMPLE 7

### HPLC Screening Assays for Substances that would Increase Peripheral or Brain Nesfatin-1

Blood samples or brain tissue samples can be taken from mice injected with various substances (such as chemical compounds, proteins, peptides or nucleic acids), and then applied to HPLC as described above. The amount of nesfatin-1 in the sample can then be measured and recorded. In one embodiment, when nesfatin-1 in the sample is found to be 20% higher than that of mice injected with saline, the substance injected in the mice would be selected as an agent for increasing peripheral or brain nesfatin-1.

### EXAMPLE 8

### Use of Plasmin or Plasminogen Activators to Inactivate Nesfatin-1

In one embodiment, plasmin or plasminogen activator (such as tissue plasminogen activator, urokinase-type plasminogen activator, streptokinase or staphylokinase) at the dose higher than 5 mg per patient per day can be i.v. administrated to patients. The blood or brain nesfatin-1 would be decreased or inactivated. The patient would have an increase in food intake, appetite, blood glucose, or body weight.

### EXAMPLE 9

### Injection of Nesfatin-1 Significantly Reduced Triglyceride, Total Cholesterol and LDL but not HDL in Blood

One hundred ug Nesfatin-1 was injected into the tail vein of ob/ob mice. Blood samples were taken 3 hours after the injection for lipid analysis. Triglyceride, total cholesterol and LDL were significantly reduced by the injection of nesfatin-1, while HDL was unaffected.

### EXAMPLE 10

### Anti-Diabetic Effect of Nesfatin-1 is Mediated by PPAR-Gamma and AMPK

GW9662, a PPAR-gamma irreversible inhibitor, was i.v. injected into the tail vein of db/db mice at the dose of 0.45 μg per gram body weight. After 30 minutes, 100 ug Nesfatin-1 was injected into the tail vein of db/db mice. Blood glucose was measured in 6 hours.

No reduction in blood glucose was found in mice injected with GW9662 prior to the injection of nesfatin-1. In contrast, without pre-treatment of GW9662, nesfatin-1 significantly reduced blood glucose in db/db mice (see above). Therefore, GW9662 fully inhibited the anti-diabetic effect of nesfatin-1 in db/db mice, suggesting that PPAR-gamma mediates the effect of nesfatin-1.

Compound C, a 5′-AMP-activated protein kinase (AMPK) specific inhibitor, was intraperitoneally injected to db/db mice at the dose of 20 mg per kg body weight. Subsequently, 100 ug Nesfatin-1 was injected into the tail vein of db/db mice. Blood glucose was measured in 6 hours.

No reduction in blood glucose was found in mice injected with Compound C prior to the injection of nesfatin-1. In contrast, without pre-treatment of Compound C, nesfatin-1 significantly reduced blood glucose in db/db mice. Therefore, Compound C fully inhibited the anti-diabetic effect of nesfatin-1 in db/db mice, suggesting that AMPK also mediates the effect of nesfatin-1.

### EXAMPLE 11

### Nesfatin-1 Analogues with Larger Molecular Weights

Since nesfatin-1 was found to affect rats neuropsychologically as evidenced by increasing anxiety and fear-related behaviors, larger-molecular-weight nesfatin-1 analogues that effectively reduce blood glucose but is prevented from penetrating blood-brain barrier (BBB) were made as follows. In one embodiment, a chemical conjugate of nesfatin-1 and albumin can be made.

### Synthesis of Albumin-Nesfatin-1 Conjugate

20 mg nesfatin-1 (0.002 mmol)was solved in 5 mL 0.1M PBS buffer (pH7.2) to give a clear solution, 4 mg (0.01 mmol) SMPT (4-succinimidyloxycarbonyl-a-methyl-[2-pyridylditho]toluene]) solved in acetonitrile with concentration 10 mg/ml was added drop-wisely into nesfatin-1 solution with rapid stirring. The mixture was kept stirring overnight at room temperature and then dialyzed against 0.1M PBS and 10 mM EDTA to remove excess reagent and to exchange the buffer. 84 mg bovine albumin (0.0013 mmol) solved in 8 mL PBS-EDTA solution was then added to the modified nesfatin-1 solution, the conjugation was quantified to measure the leaving group pyridine-2-thione, which has an absorption maximum at 343 nm, using a spectrophotometer. After 48 hours reaction at room temperature, the excess pyrinde-2-thione groups were quenched with 0.4 mg cystein. The conjugate was obtained after the size exclusion chromatography to remove the free nesfatin-1 and the modified nesfatin-1. During the whole reaction, 10% SDS-PAGE Gel was used to monitor and evaluate the conjugate reaction.

Even though the SMPT was in 4 fold molar excess, about 20-30% free nesfatin-1 was detected by analytical HPLC in the modified solution. The conjugate reaction was mostly stopped after 48 hours, since the absorption at 343 nm had not obvious increment. SDS-Page gel also showed there has not too much change after 48 hours reaction. The yield of the albumin-nesfatin-1 conjugate is about 50-60% estimated from the gel.

### EXAMPLE 12

### Large Molecular Weight Nesfatin-1 Analogue Reduces Blood Glucose Without Penetration of Blood-Brain Barrier

The albumin-nesfatin-1 conjugate was labeled with 1125 and intravenously injected to db/db mice and C57B1/6J mice. Brain samples were taken at 0, 3, 5, 10, 30 and 60 minutes after the injection to measure its radioactivity using a gamma counter. Equal amount of radioactive NaI¹²⁵ was used as positive control.

The conjugate of albumin-nesfatin-1 (250 μg) was injected into the tail vein of db/db mice. Blood glucose was measured in 6 hours. No radioactivity was detected in the mouse brain injected with I¹²⁵-labeled albumin-nesfatin-1 conjugate at any time points. In contrast, the radioactivity was detected in the mouse brain injected with NaI¹²⁵ at 3-30 minutes and decreased over time. Thus, these results indicated that the albumin-nesfatin-1 conjugate did not penetrate blood-brain barrier to enter the brain from circulation. In contrast, iv injection of conjugate of albumin-nesfatin-1 significantly reduced blood glucose in db/db mice with 6 hours.

### REFERENCES

1. Krystosek, A. & Seeds, N. W., Plasminogen activator release at the neuronal growth cone. Science 213 (4515), 1532-1534 (1981).
2. Moonen, G., Grau-Wagemans, M. P., & Selak, I., Plasminogen activator-plasmin system and neuronal migration. Nature 298 (5876), 753-755 (1982).
3. Tsirka, S. E., Rogove, A. D., Bugge, T. H., Degen, J. L., & Strickland, S., An extracellular proteolytic cascade promotes neuronal degeneration in the mouse hippocampus. J Neurosci 17 (2), 543-552 (1997).
4. Wang, N., Zhang, L., Miles, L., & Hoover-Plow, J., Plasminogen regulates pro-opiomelanocortin processing. J Thromb Haemost 2 (5), 785-796 (2004).
5. Hoover-Plow, J., Wang, N., & Ploplis, V., Growth and behavioral development in plasminogen gene-targeted mice. Growth Dev Aging 63 (1-2), 13-32 (1999).
6. Chua, S. C. et al., Phenotypes of mouse diabetes and rat fatty due to mutations in the OB (leptin) receptor. Science 271 (5251), 994-996 (1996).
7. Oh-I, S. et al., Identification of nesfatin-1 as a satiety molecule in the hypothalamus. Nature 443 (7112), 709-712 (2006).
8. Shimizu, H. et al., Peripheral administration of nesfatin-1 reduces food intake in mice: the leptin-independent mechanism. Endocrinology 150 (2), 662-671 (2009).
9. Price, T. O., Samson, W. K., Niehoff, M. L., & Banks, W. A., Permeability of the blood-brain barrier to a novel satiety molecule nesfatin-1. Peptides 28 (12), 2372-2381 (2007).
10. Pang, P. T. et al., Cleavage of proBDNF by tPA/plasmin is essential for long-term hippocampal plasticity. Science 306 (5695), 487-491 (2004).
11. Zhang, L., Gong, Y., Grella, D. K., Castellino, F. J., & Miles, L. A., Endogenous plasmin converts Glu-plasminogen to Lys-plasminogen on the monocytoid cell surface. J Thromb Haemost 1 (6), 1264-1270 (2003).
12. Pan, W., Hsuchou, H., & Kastin, A. J., Nesfatin-1 crosses the blood-brain barrier without saturation. Peptides 28 (11), 2223-2228 (2007).
13. Merali, Z., Cayer, C., Kent, P., & Anisman, H., Nesfatin-1 increases anxiety- and fear-related behaviors in the rat. Psychopharmacology (Berl) 201 (1), 115-123 (2008).
14. Selvarajan, S., Lund, L. R., Takeuchi, T., Craik, C. S., & Werb, Z., A plasma kallikrein-dependent plasminogen cascade required for adipocyte differentiation. Nat Cell Biol 3 (3), 267-275 (2001).
15. Zheng, F. et al., Development of albuminuria and glomerular lesions in normoglycemic B6 recipients of db/db mice bone marrow: the role of mesangial cell progenitors. Diabetes 53 (9), 2420-2427 (2004).
16. Hastings, G. A. et al., Neuroserpin, a brain-associated inhibitor of tissue plasminogen activator is localized primarily in neurons. Implications for the regulation of motor learning and neuronal survival. J Biol Chem 272 (52), 33062-33067 (1997).
17. Cota, D. et al., Hypothalamic mTOR signaling regulates food intake. Science 312 (5775), 927-930 (2006).

What is claimed is:

1. A method of treating a subject having diabetes, comprising the step of administering to the subject an effective amount of an agent that leads to increased peripheral nesfatin- 1.

2. The method of claim 1, wherein the agent is administered intravenously, subcutaneously, or orally.

3. The method of claim 1, wherein the agent is nesfatin-1 or a portion thereof.

4. The method of claim 3, wherein the nesfatin-1 is a conjugated nesfatin-1 with increased molecular weight.

5. The method of claim 4, wherein the conjugated nesfatin-1 reduces blood glucose without penetration of blood-brain barrier.

6. The method of claim 1, wherein the agent is a plasmin inhibitor.

7. The method of claim 6, wherein the plasmin inhibitor is aprotinin, AMCA, EACA or their analogues.

8. The method of claim 1, wherein the subject is having type II diabetes or type I diabetes.

9. The method of claim 8, wherein when the subject is having type I diabetes, the method further comprises the step of administering insulin to the subject.

10. A transgenic diabetic or obese rodent comprising homozygous plasminogen gene disruption, wherein the transgenic rodent exhibits reduced body weight or reduced blood glucose as compared to a diabetic or obese rodent not having the plasminogen gene disruption.

11. The transgenic rodent of claim 10, wherein the rodent further comprises homozygous leptin gene disruption or homozygous leptin receptor gene disruption.

12. The transgenic rodent of claim 10, wherein the rodent is a mouse.

13. A method of reducing triglyceride, total cholesterol or LDL in blood, comprising the step of administering to a subject an effective amount of an agent that leads to increased peripheral nesfatin-1.

14. The method of claim 13, wherein the agent is nesfatin-1 or a portion thereof, a conjugated nesfatin-1 with increased molecular weight, or a plasmin inhibitor.

15. A method of screening for an agent that would increase peripheral or brain nesfatin-1, comprising the steps of:
(a) administering a candidate agent to a subject;
(b) obtaining blood samples or brain tissue samples from the subject; and
(c) determining the amount of nesfatin- I in the samples, wherein an increased amount of nesfatin-1 as compared to that in samples obtained from subject treated with a control substance would indicate that the candidate agent would increase peripheral or brain nesfatin-1.

16. The method of claim 15, wherein the amount of nesfatin-1 is determined by a HPLC assay.

17. The method of claim 15, wherein the subject is the rodent of claim 10.
